(19) United States
(12) Patent Application Publication
(10) Pub. No.: US 2020/0139064 A1
(43) Pub. Date: May 7, 2020

(54) Connector Device

(71) Applicant: Christopher Hill, Cottesloe (AU)

(72) Inventor: Christopher Hill, Cottesloe (AU)

(21) Appl. No.: 16/312,649

(22) PCT Filed: Jul. 7, 2017
(86) PCT No.: PCT/AU2017/050704

§ 371 Date: Dec. 21, 2018

(30) Foreign Application Priority Data
Jul. 9, 2016 (AU) 2016902698

(51) Int. Cl. A61M 16/04; A61M 16/08

(52) U.S. Cl. CPC A61M 16/0488; A61M 25/0102; A61M 2202/0208; A61M 16/0816

(57) ABSTRACT

A connector device 10 for releasably connecting a bougie 18 or stylet to an endotracheal (intubation) tube (ETT) 12 has a body portion 14 having an opening 16 provided as a longitudinal bore or channel. The connector device can be adjusted to grip the bougie or stylet. The connector device can have passages 40, 41 and a port 42 for supply of gas into or from the ETT. The connector device can include clamping means 30, 32 to clasp the exterior of the ETT to internally grip the bougie or stylet within the ETT. The connector device can grip the bougie or stylet by insertion of the connector device into an open connection end of the ETT. The connector device can form part of the bougie or stylet or be attached thereto, such as by moulding or bonding or other attachment means.

### FIELD OF THE INVENTION

The present invention relates to a connector device having application in the procedure of introducing an object into or through a passageway of a body, such as introducing an endotracheal tube into an airway.

### BACKGROUND TO THE INVENTION

It is known in medical procedures to introduce an object into or through a body passageway for purposes of diagnosis or treatment. An example of such a procedure is the introduction of an endotracheal tube (ETT) into the trachea, usually for purposes of maintaining an open airway or to provide a conduit through which to administer oxygen, medicine or anaesthesia, or medical equipment such as suction apparatus.

Endotracheal intubation is often carried out with the aid of a tube introducer, commonly referred to as a ‘bougie’. Intubation with the aid of a bougie is typically carried out by an Operator and Assistant, working together and performing the following steps:
1. The Operator performs laryngoscopy;
2. The Assistant hands the bougie to the Operator;
3. The Operator passes the bougie through the glottis and into the trachea (a procedure sometimes termed ‘bouginage’ or ‘bougienage’);
4. The Assistant places an ETT over an external or exposed end of the bougie;
5. The Operator holds the bougie as the ETT is advanced over the bougie until an end of the bougie emerges from an end of the ETT;
6. The Assistant holds the portion of the bougie that extends outwardly from the end of the ETT;
7. The Operator releases the bougie;
8. The Operator grasps the ETT and advances it along the bougie, through the glottis and into the trachea;
9. The Assistant removes the bougie; and
10. The ETT is secured in position.

Typically, the bougie has an external diameter that is smaller relative to an internal diameter of the ETT. When the bougie is located inside the ETT, the bougie and ETT are not held in fixed contact with or relative to each other. The bougie is able to move independently relative to the ETT in both longitudinal and rotational planes. This can result in diminished control of the bougie and/or ETT, potentially increasing complexity, duration and risk of the procedure and reducing likelihood of success of the procedure.

Some methods have been attempted to counter the movement of the bougie relative to the ETT. In one method, an end of the bougie is bent around 270° and is gripped together with the ETT, providing at least some degree of steadiness of the ETT relative to the bougie. In another method, the external end of the bougie is bent 180° and inserted into the external open end of the ETT. This forms a ‘D’ loop and similarly provides some stability between the ETT and bougie.

These methods add undesirable degrees of handling complexity to the procedure. Further, they can result in deformation of the bougie, impairing the intended and further use of the bougie in the procedure and for reuse in other procedures after sterilising (in the event the bougie is not a single use bougie).

It is therefore desirable to secure or otherwise limit movement of a bougie within or relative to an ETT when performing endotracheal intubation to achieve improved handling procedures and optionally better manipulative control of the ETT and bougie.

It is further desirable that such improved handling or control be achieved without requiring deformation of the bougie.

### SUMMARY OF THE INVENTION

With the abovementioned in mind, according to a first aspect of the present invention there is provided a connector device comprising: a body portion having an opening for receiving a bougie; and a tube engaging portion; wherein a bougie or stylet locates into the opening of the body portion and the tube engaging portion engages with a tube.

Preferably, the connector device further includes securing means to limit movement of the bougie or stylet in longitudinal and/or rotational planes relative to the tube. The securing means may also limit movement of the bougie or stylet relative to the connector device.

The tube is typically a tube used in medical procedures such as an endotracheal tube (ETT), having a connector or connector end that engages with the tube engaging portion of the connector device.

Preferably, the opening of the body portion is shaped to receive a portion of the bougie or stylet, such as a longitudinal mid-portion.

The bougie or stylet may be received in the opening of the body portion of the connector device such that a portion of the bougie extends outwardly of the body portion. The bougie or stylet with the connector device attached thereto may then be introduced into the ETT through an open end thereof and the bougie or stylet is advanced into the connector end of the ETT until the connector end of the ETT engages with the connector device when a required or preferred portion of the bougie or stylet extends outwardly from an opposing end of the tube.

The bougie may be rotationally adjusted in the tube until the bougie or stylet is in a desired position relative to the tube.

Rotational orientation of the bougie or stylet typically orients an angled or Coude tip of the bougie or stylet in a ventral or anterior direction. The bougie or stylet may be longitudinally adjusted in position relative to the connector device and/or the tube.

Preferably, when the bougie or stylet is appropriately positioned in the tube, the securing means can be operated or activated to hold the bougie or stylet in position relative to the connector device and tube, limiting movement of the bougie or stylet in longitudinal and/or rotational planes relative to the tube.

The position of the bougie or stylet about a longitudinal axis may be adjusted relative to the connector device and/or the tube prior to or after the engagement with the connector device.

The position of the bougie or stylet about the rotational axis can be adjusted prior to or after engagement of the tube engaging portion of the connector device with the connector end of the ETT.

The tube engaging portion engages or mates with, over or into the end of the tube. In at least one embodiment, the tube engaging portion engages or mates with a connector portion of the tube.

Preferably, the securing means is releasable, enabling the device to be disengaged from the bougie or stylet. The bougie or stylet, or bougie/stylet and device, can then be removed from the tube once the tube has been appropriately positioned.

Advantageously, the device permits the bougie or stylet to be passed through the tube, whereupon the bougie and/or tube can be adjusted relative to each other in longitudinal and rotational planes before securing the bougie or stylet and tube in the desired position. The tube and bougie/stylet, connected by the connector device, can then be directed and manipulated essentially as a single unit.

According to a further aspect of the invention, there is provided a connector device comprising a first clamp portion and a second clamp portion, the first and second clamp portions being connected and movable relative to each other between an open position and a closed position, wherein the first and second clamp portions are adapted to hold a tube therebetween when in the closed position and apply a force to the tube, whereby an inside surface of the tube grips a bougie or stylet located inside the tube, limiting movement of the bougie or stylet relative to the tube.

Preferably, the connector further includes releasable securing means to hold the first and second clamp portions in the closed position.

The first and/or second clamp portions may have an opening for receiving the tube.

Advantageously, force applied to the tube by the first and second clamp portions in the closed position compresses the tube inwardly, causing an internal surface of the tube to press against the bougie or stylet located therein and limiting longitudinal and/or rotational movement of the bougie or stylet within the tube.

The connector device may include at least one vent or port, such as for connection of a tube to deliver gas into or through the connector device or to ventilate gas out of the connector device.

The connector device may include a channel along and into a length of the connector device leading to a conduit or passage for receiving the bougie or stylet.

The connector device may include one or more septations (septa) or divisions forming one or more respective passages through or one or more chambers within the connection device.

The connector device may include at least one grip for assisting removal of the connector device from the ETT. The grip may include a loop, handle, projection and/or textured surface.

The connector may be incorporated with a bougie or stylet. The connector device may be moulded with or moulded to the bougie or stylet or adhered or bonded to the bougie or stylet.

The connector device may include a lever actuated mechanism for use in gripping and/or releasing the bougie or stylet.

The connector device may include a cam mechanism wherein a cam engages with the bougie or stylet to retain the bougie or stylet to the connector device.

It wil be appreciated that the present invention is applicable to retaining a stylet within an ETT tube rather than a bougie. Consequently, reference to bougie also encompasses a stylet.

For applications involving a stylet, the stylet may be shaped to conform the ETT to a desired curvature, and the connector device is used to hold the stylet and ETT together.

The connector may include a cam mechanism wherein a cam engages with the bougie or stylet to retain the bougie or stylet to the connector device.

The connector device may include an engaging portion to engage with the tube end of the ETT. The connector device engaging portion may fit over or into or both over and into the tube end of the ETT, and the connector device provides a passage for the bougie or stylet to pass into the ETT.

### BRIEF DESCRIPTION OF THE DRAWINGS

Further advantages and details of the present invention will become apparent with the benefit of the following detailed description of embodiments and having reference to the accompanying drawings in which:

FIG. 1 is a side view of a first embodiment of a connector device in accordance with an embodiment of the present invention;

FIG. 2A is a side perspective and FIG. 2B a cross-section view of a connector device in accordance with another embodiment of the invention;

FIG. 2C is a side section view of a connector device in position within a connector end of an ETT according to an embodiment of the present invention;

FIG. 2D shows an embodiment of the connector device having two halves hinged at a join;

FIG. 3 is a side perspective view of a connector device in accordance with a further embodiment of the present invention;

FIGS. 4a to 4c show a connector device in accordance with a further embodiment of the present invention;

FIGS. 5a to 5c show steps in use of the connector device of FIGS. 4a to 4c;

FIGS. 6a to 6c show steps of use of an alternative embodiment of the present invention;

FIG. 7 shows a connector device according to an embodiment of the present invention provided on a bougie;

FIGS. 8a and 8b show a septated or internally divided connector device according to a further embodiment of the present invention;

FIGS. 9a to 9b show a connector device according to an embodiment of the present invention including a vent connection;

FIGS. 10 to 13 show various alternative embodiments of the connector device of the present invention;

FIG. 14 shows a view of a connector device according to an embodiment of the present invention received into a connection end of an ETT;

FIGS. 15 to 16 show steps in receiving a bougie into a connector embodying the present invention, and then receiving the connector device into the open connector end of an ETT; and

FIGS. 17 to 22 show alternative embodiments of a connector device of the present invention retaining a bougie or stylet within a connector end of an ETT.

FIG. 23 shows an alternative embodiment of the connector of the present invention fitting directly to an end of the tube of the ETT.

### DESCRIPTION OF PREFERRED EMBODIMENT(S)

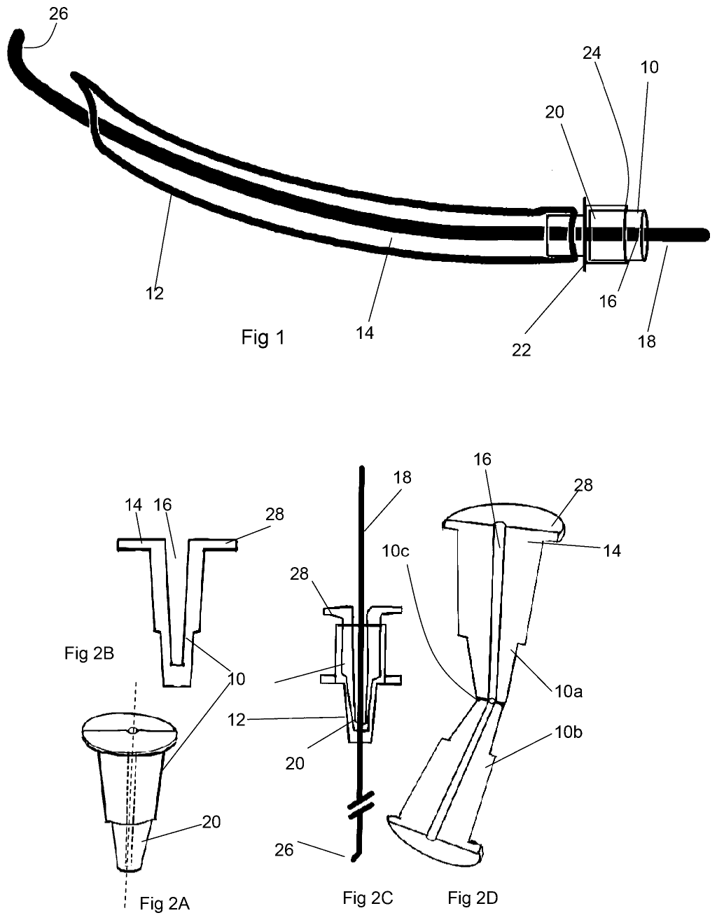

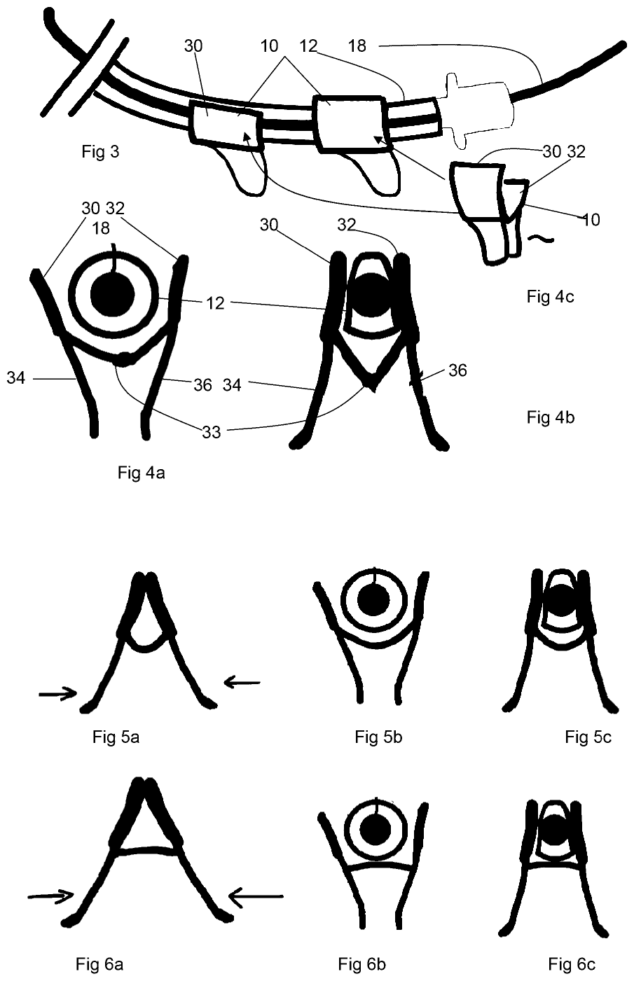

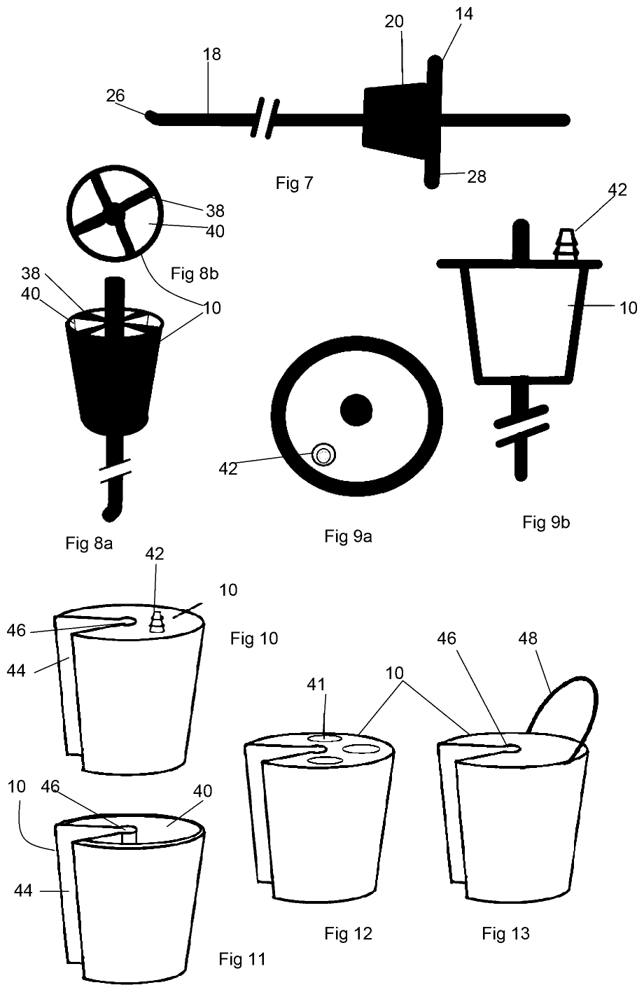

Referring initially to FIG. 1, there is shown a connector device 10 for use in procedures requiring introduction of an object into or through a body passageway, such as the introduction of an endotracheal tube (ETT) 12 into the trachea.

The connector device 10 includes a body portion 14 having an opening 16 located therein.

In the embodiment shown, the body portion 14 is substantially cylindrical or disc-like. The opening 16 is provided as a longitudinal bore or channel. The opening 16 is disposed centrally through a longitudinal length of the cylinder or disc of the body portion 14. However, it should be understood that the opening 16 can be located through the body portion 14 in alternative arrangements. For example, the opening 16 can extend through the body portion 14 off-centre, or extend from a side of the body portion 14 towards an end thereof.

Alternative locations of the opening 16 may be preferred if the body portion 14 has an alternative shape or configuration, for example, substantially wedge-shaped.

The opening 16 has dimensions and configuration that can accommodate at least a portion of a bougie 18. That is, the opening 16 has a diameter that is comparable with, or slightly larger relative to diameter of a bougie 18.

The diameter of the opening 16 is large enough to permit the bougie 18 to pass through by exertion of a small amount of force and to be rotated within the opening 16. Ideally, the diameter of the opening 16 is sufficiently small to minimise lateral movement of the bougie 18 in the opening 16. The bougie 18 can be introduced into the opening 16 and located therein such that a length of the bougie 18 extends outwardly from the opening 16 and body portion 14 at opposing open ends thereof.

The connector device 10 further includes a tube engaging portion 20. In the embodiment shown in FIG. 1, the tube engaging portion 20 extends outwardly from an end of the body portion 14. The tube engaging portion 20 is substantially cylindrical in shape, having a diameter that is smaller relative to that of the body portion 14. The diameter of the tube engaging portion 20 is comparable to a diameter of an opening of a tube connector portion 22 of an ETT 12, enabling the tube engaging portion 20 to mate with the tube connector portion 22. In the embodiment shown, the tube engaging portion 20 connects with the tube connector portion 22 in a male-to-female configuration, though the connection may also be female-to-male.

The tube engaging portion 20 has a central opening 24, extending from and in communication with the opening 16 of the body portion 14. The bougie 18, once introduced into the device 10, extends through the opening 16 of the device 10 with a distal end 26 extending outwardly from an end of the tube engaging portion 20. Typically, the distal end 26 of the bougie 18 is an angled, curved or coudie tip.

In this embodiment, the bougie 18 is passed through the body portion 14 of the device 10 by introducing a distal end 26 of the bougie 18 into the opening 16 and advancing the bougie 18 towards the open end of the tube engaging portion 20. The bougie 18 is positioned relative to the device 10 to allow sufficient length from the device 10 to distal end 26 of the bougie 18.

The bougie 18 with connector device 10 thereon is introduced into the ETT 12 until the tube engaging portion 20 abuts the connector portion 22 of the ETT 12. The position of the bougie 18 can be adjusted about a rotational axis as required prior to mating of the tube engaging portion 20 and connector portion 22. Alternatively, the tube engaging portion 20 and connector portion 22 can first be mated by inserting the connector portion 22 into the tube engaging portion 20 and press fitting therein. The position of the bougie 18 can then be adjusted about the rotational axis as required by the operator.

Fastening of the device 10 to the ETT 12 allows for the bougie 18 to be held in a desired position relative to the ETT 12 whilst permitting rotational adjustment of the bougie 18 to orient the Coude tip 26 of the bougie 18 in a ventral or anterior direction. With the bougie 18 held in position by the device 10, the bougie 18 and ETT 12 can essentially be manipulated and directed as a single unit.

It is preferred that the connector device 10 is provided with a securing means to secure the bougie 18 in position relative to the device 10 in longitudinal and/or rotational planes. The securing means may take any number of suitable forms and may be as simple as a friction fit of the bougie 18 inside the opening 16. Further securing means will be described below with reference to alternative embodiments of the invention.

Referring now to FIGS. 2A to 2D, there is shown a second embodiment of the connector device 10, where like numerals refer to like features of the first embodiment. This second embodiment also includes a body portion 14 with a opening 16 extending therethrough as a central longitudinal bore. The body portion 16 is substantially or generally tapered or frusto-conical in form, narrowing inwardly as it extends towards the tube engaging portion 20.

The body portion 14 has a flange 28 extending outwardly from an end of the body portion 14 distal to the tube engaging portion 20. The flange 28 is useful for grasping by the operator.

The tube engaging portion 20 is similarly tapered or frusto-conical in form, narrowing inwardly towards an open end distal to the body portion 14. The shape and size of the tube engaging portion 20 is comparable to a standard connector portion 22 of an ETT 12, allowing mating of the tube engaging portion 20 with connector portion 22.

In this embodiment, body portion 14 and tube engaging portion 20 are integrally formed or moulded. The body portion 14 and tube engaging portion 20 are together formed of two halves 10a, 10b, the halves being mirror images of each other along a central cross section. Optionally, the two halves can be joined 10c to each other at an end thereof. Alternatively, the two halves 10a, 10b can be provided as two separate, and can be substantially identical components.

The bougie 18 is locatable in the opening 16 essentially as described above. The bougie 18 can be located in the opening 16 while the halves of the body portion 14 are separated from each other or after the two halves are placed alongside each other. Once the bougie 18 is appropriately placed in the opening 16, the two halves of the body portion 14 are closed together, retaining the bougie 18 inside. The distal end 26 of the bougie 18 is then introduced into the ETT 12 and passed therethrough as described above, until the tube engaging portion 20 abuts the connector portion 22 of the ETT 12. Once the bougie 18 has been rotationally and/or longitudinally adjusted to the satisfaction of the operator, the tube engaging portion 20 is mated with the connector portion 22, creating the single unit of device 10, bougie 18 and ETT 12.

The connector device 10 may include the body portion 14 being substantially frusto-conical in shape, with a central longitudinal bore opening 16. In this embodiment, the tube engaging portion 22 is integrally formed with the body portion 14, whereby the tube engaging portion 22 is the tapered end of the body portion 14.

The body portion 14 and tube engaging portion 20 can be formed from two halves, connected to each other along an edge thereof, ideally by a hinge arrangement. For example, along a longitudinal join along a side of the connector device or along a transverse join at an end of the connector device. Once the bougie 18 is introduced into the opening 16 and appropriately positioned along the longitudinal plane, the two halves of the body portion 14 are brought together, enclosing the bougie 18 therein. The bougie 18 is secured in place by compression fit. Optionally, the opening 16 can have a roughened surface to assist in securing the bougie 18 therein.

The distal end 26 of the bougie 18 is introduced into the ETT 12 as described above until the base of the body portion 14, or tube engaging portion 20, abuts the connector portion 22 of the ETT. The connector portion 22 and tube engaging portion 20 are mated following rotational adjustment of the bougie 18 as required.

Referring now to FIGS. 4a to 4c, there is shown a further embodiment of the connector device 10, including first clamp portion 30 and second clamp portion 32. The first and second clamp portions 30, 32 are connected to each other. In the embodiment shown in the Figures, the first and second clamp portions 30, 32 are connected by a connecting portion 33, traversing between the clamp portions 30, 32. The first and second clamp portions 30, 32 may be hingedly connected, including by a hinge or spring connection.

The first and second clamp portions 30, 32 are movable relative to each other between an open position (FIG. 4a) and a closed position FIG. 4b).

When in the closed position, the first and second clamp portions 30, 32 are adapted to apply force to the ETT tube 12, compressing the tube 12 inwardly. In this manner, the inside of the tube 12 presses against the bougie 18 located inside the tube 12, restricting or preventing movement of the bougie 18 therein.

The first and second clamp portions 30, 32 each have sufficient surface area contacting the tube 12 to avoid damage of the tube 12 when the tube 12 is compressed inwardly. One or both of the first and second clamp portions 30, 32 may have a shape or configuration adapted to receive the tube 12. As an example, at least a part of the first and/or second clamp portions 30, 32 may be substantially cylindrical or semi-cylindrical to accommodate shape and configuration of the tube 12.

Each clamp portion 30, 32 can extend into a grip portion 34, 36. The grip portions 34, 36 operate as a lever about the connecting portion 33. That is, a user pushes each grip portion 34, 36 towards the other in a pinching action to move the clamp portions 30, 32 to the open position. Upon release, the grip portions 34, 36 are biased apart from each other by the connecting portion 33, returning the clamp portions 30, 32 to the closed position.

The first and second clamp portions 30, 32 are moved into the closed position about the tube 12 once the bougie 18 has been suitably located in the tube 12, as described above, holding the bougie 18 in place within the tube 12. The clamp portions 30, 32 can be further held in the closed position by securing means, such as a clip. However, in the embodiments shown, clamp portions 30, 32 can be sufficiently held in the closed position by the force applied by the connector portion 33, operating as a biasing means.

Once the combined tube 12/bougie 18 has been appropriately located in the airway, the operator can unfasten the securing means, such as with their thumb, releasing the securing means and allowing the first and second clamp portions 30, 32 to be moved into the open position. Alternatively, the operator can unfasten the device 10 from about the tube 12 and bougie 18 by pressing the grip portion 34, 36 towards each other, thereby moving the clamp portions 30, 32 apart from each other and releasing the tube 12/bougie 18.

In each of the embodiments described above, the device 10 includes securing means to secure the bougie 18 in position relative to the device 10 in longitudinal and/or rotational planes. Various securing means are envisaged, including but not limited to a locking mechanism or means, compression or friction fit inside the opening 16, clip, lever, spring bias, guillotine grip or roller ball mechanism. Securing means can be suitably located, such as on an outer surface of the body portion 14 and moved from a first position which allows movement of the bougie 18 within the device 10, to a second position in which the bougie 18 is held in place within the opening 16.

In each embodiment described above, the device 10 advantageously assists in conducting procedures such as intubation. The device 10 can be coupled to the ETT 12 and bougie 18 during preparation of airway equipment in advance of an intubation. The device 10 permits the bougie 18 to be passed through the ETT 12 such that a sufficient length extends out from a distal end of the ETT 12. The bougie 18 can be adjusted rotationally to orient the Coude tip of the bougie in an appropriate direction. Activation of the securing means holds the bougie 18 in the desired position relative to both device 10 and ETT 12 and allows the ETT 12 and bougie 18 to be operated essentially as a single device.

Using the device 10 of the present invention, intubation steps 2-7 described in the background of this application can be eliminated. Further, intubation can potentially be performed by a single operator, instead of requiring both operator and assistant.

The enlarged portion of the connector is arranged and configured to engage with the connector portion of the ETT. For example, the connector may be wedged or tapered in shape to facilitate engagement/removal with/from the ETT. However, a wedge or tapered form is not a requirement. The passages can pass completely through the connector device. Chambers can be formed by having a closed end of the connector device.

As shown in FIGS. 7 and 14, the bougie may integrally include or have attached thereto the connector device before insertion of the bougie into the ETT. For example, the connector device may be moulded to the material of the bougie or be formed with the bougie, of may be adhered to or otherwise bonded or attached to the bougie.

As shown in FIGS. 8a, 8b and 14, the connector device 10 can include at least one separation or division 38 (such as by one or more walls, surfaces or membranes) of passages or chambers 40 through or in the connector for free flow of gas into/from the ETT. For example, sometimes it is useful to see the ETT tube ‘fogging up’ to help confirm placement in the airway with spontaneous airflow or assisted ventilation. The bougie may or may not be in situ in the ETT at this stage.

As shown in FIGS. 15 and 16, the connector device can include a port 42 to attach tubing, such as for an oxygen supply or for ventilation or suction. The port can be continuous with the lumen of the bougie or can have a conduit/passage connecting with the larger diameter of the ETT, such as for oxygenation during intubation. The bougie has a lumen extending through its length and is open at both ends as standard and can deliver oxygen if connected. Both ends of the enlarged portion can be sealed to facilitate gas flow if connected. There may need to be a ‘release’ vent incorporated to prevent barotrauma if the end becomes blocked/misplaced when high flow (e.g. 15L per minute) is being delivered.

The enlarged portion can be at a fixed position on the length of the bougie. There can be an advantage in being able to move the connector device along the length of the bougie to adjust the length of exposed bougie beyond the ETT tip.

A grip 48 can be provided, which can include a loop, handle or extension/projection from the flange or body of the connector device to assist removal of the connector device from the ETT connection end.

Any one or more embodiment of the present invention can incorporate a port (such as for the supply of oxygen into the ETT or ventilation from the ETT)

Alternatively, or in addition, one or more conduits/passages for free flow of gas can be provided through the connector device to communicate gas into the ETT and around the bougie within the ETT. The septations (septa) 38 forming one or more passages 40 (such as perforations 41) can be provided in the connector device.

According to one or more alternative embodiments of the present invention, a plug or hollowed embodiment of the connector may be applied to the bougie at a desired position along the bougie once the bougie has been compared to the endotracheal tube (ETT) for length comparison. A user can ‘size up’ the bougie against the ETT before the bougie is inserted into the ETT, and then apply the connector to the bougie, with the connector gripping the bougie in the desired place along its length and inserting the bougie into the ETT with the connector making a fit, preferably a compression or interference fit with the end of the ETT tube.

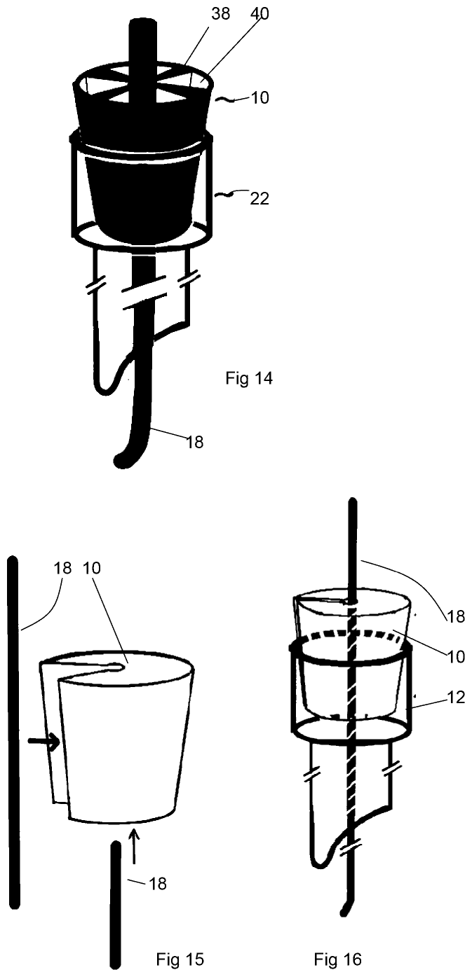

As shown in FIGS. 10 to 13, the connector device 10 can include a channel 44 leading to conduit 46 passing through the length of the connector device to receive the bougie 18. Once the bougie is inserted via the channel into the conduit/passage, the connector device clamp around the bougie (such as by the connector device being inserted into the open connection end of the ETT and pressure on the side wall of the connector device against the internal wall of the ETT providing a reaction force to squeeze the connector device to grip the bougie, as shown, for example, in FIGS. 15 and 16.

Alternatively, the connector device can be slid over or onto the bougie so it is loosely attached but can easily be moved along the length or rotated on the bougie. Then, once the bougie is passed into the ETT with the desired distal length exposed, the connector can be slid down to engage the connector end of the ETT and be retained in position.

A further alternative embodiment accommodates the connector being applied into the connector end of the ETT, then the bougie length within or extending from the ETT can be adjusted, and then the connector is ‘pressed home’ in the connector end of the ETT to secure the bougie in position relative to the ETT.

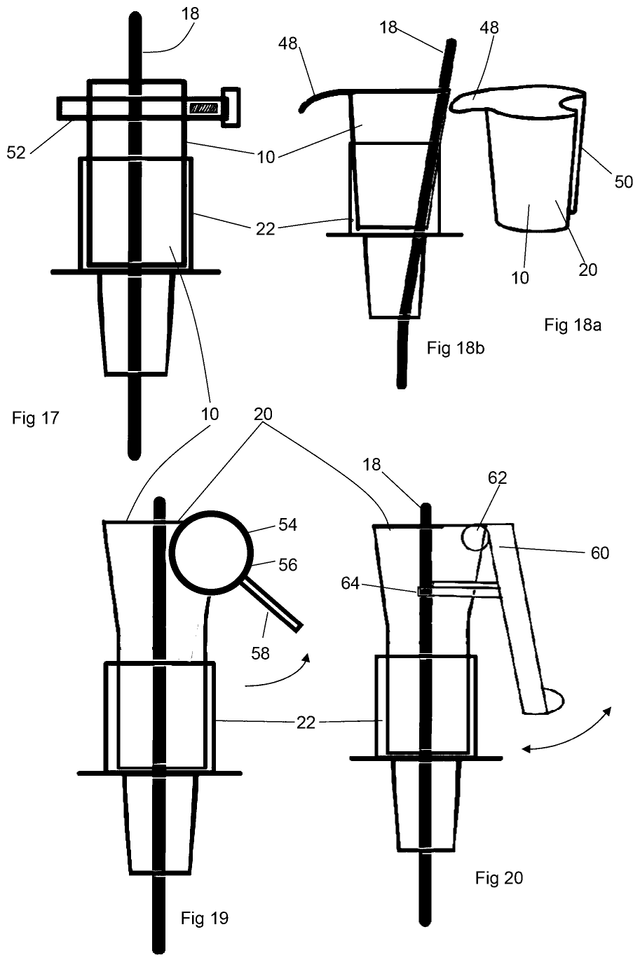

FIG. 17 shows a version of the connector device 10 having a guillotine type clamp 52 to apply pressure to the connector body or to the bougie to retain the bougie in fixed relation to the connector device and the ETT.

FIG. 18a shows an alternative form of the connector device 10 in the form having of a wedge or tapered body having a groove or channel 50 extending longitudinally along a side thereof. As shown in use in FIG. 18b, the connector device 10 fits into an open end of the connector end 22 of the ETT and traps the bougie 18 within the groove or channel 50 and against the interior side wall of the connector end. The groove or channel 50 may partially or completely surround the portion of the bougie present within the connector device.

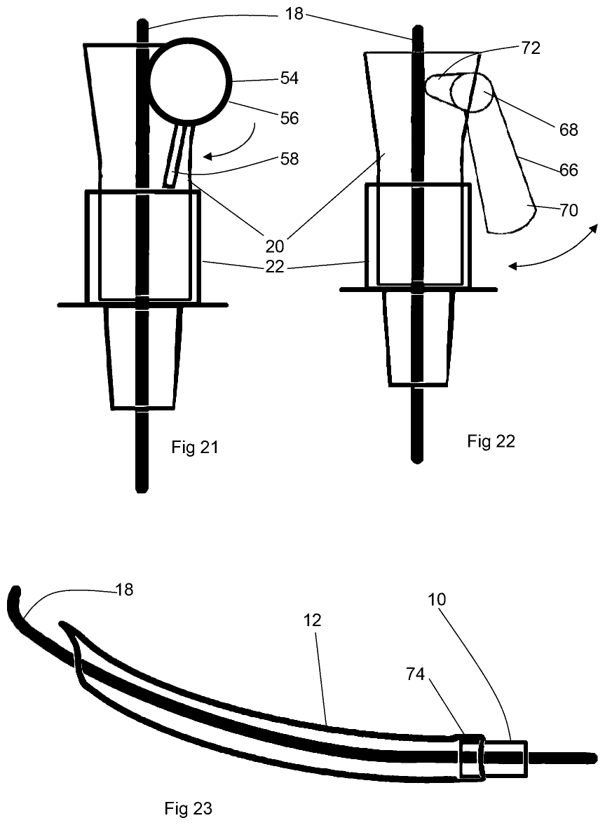

FIGS. 19 and 21 show two stages of a lever action clamping version of the connector device 10. FIG. 19 shows the lever acting mechanism 56 with a lever 58 in a release position and the cam device released from engagement with the bougie, such that the bougie is free to move within the connector device 10. FIG. 21 shows the lever 58 in an engaged position such that the cam device rotates to engage with the bougie and hold the bougie within the body of the connector device retraining movement of the bougie relative to the connector device and the ETT into which the connector device is received.

FIG. 20 shows an alternative lever acting mechanism 60 wherein a lever rotates about a pivot 62 when actuated, causing a retainer 64 to retain the bougie within the connector device.

FIG. 22 shows an alternative embodiment of the connector device 10. A lever actuated mechanism 66 includes a lever 70 pivoting about a pivot 68 which releases or engages a cam member 72 with the bougie depending on the direction that the lever is manually moved.

A further embodiment of the device 10 is provided wherein the body portion 14 is provided as two segments 14a, 14b, each having a flange 28a, 28b extending outwardly from an end distal to the tube engaging portion 20. A first segment 14a is static relative to the tube engaging portion 20 and may be integrally formed therewith. The first segment 14a has an opening 16 to receive the bougie 18. The second segment 14b is slidably moveable relative to the first segment 14a and tube engaging portion 20.

The tube engaging portion 20 is substantially frusto-conical in shape and is arranged to receive a wedge portion 15 of the second segment 14b. The wedge portion 15 tapers inwardly as it extends away from the flange 28b. The wedge portion 15 is arranged to sit inside the tube engaging portion 20, contiguous to the first segment 14a. The wedge portion 15 is arranged to slide longitudinally relative to the tube engaging portion 20 and first segment 14a of the body portion.

Sliding action of the wedge portion 15 can be enabled by a user placing a digit, such as the forefinger, under the flange 28a of the first segment 14a, and sliding the second segment 14b and hence wedge portion 15 down by pressing down on the flange 28b of the second segment 14b with another digit, such as the thumb. Downward sliding of the wedge portion 15 presses the wedge portion 15 against the first segment 14a, providing a compression fit to hold the bougie 18 in place in the opening 16.

The second segment 14b and wedge 15 can be slid upwardly by placing a thumb or other digit under the flange 28b and pressing upwardly. This upwards movement disengages the wedge portion 15, allowing the bougie 18 to be moved within the device 10 or removed therefrom.

Second segment 14b and wedge portion 15 is thus slidable between a locked and open position. This enables easy release to allow alteration in the length of exposed bougie 18 during a procedure by unlocking to slide the bougie 18 in or out. This embodiment advantageously provides a quick release of the bougie 18 for removal or length adjustment.

The connector device can include an engaging portion to engage with the tube end of the ETT, such as the connector device engaging portion fitting over or into, or both over and into, the tube end of the ETT, and the connector device provides a passage for the bougie or stylet to pass into the ETT.

As shown in FIG. 23, the connector device can be connected directly to an end 74 of the ETT tube.

Using the device 10 of the present invention, coupled with bougie 18 and ETT 12 as described above, steps of intubation can be reduced to the following:
1. The Operator performs a laryngoscopy;
2. The operator holds the ETT 12 and bougie 18, connected by the device 10 as described above and directs the distal end 26 of the bougie 18 extending outwardly from the ETT 12 through the glottis;
3. The Operator advances the ETT/bougie/device unit into the trachea;
4. Upon locating the ETT into the trachea, the device 10 is released or unsecured from the bougie 18 and ETT 12;
5. The bougie 18 is removed from the ETT 12; and
6. The ETT 12 is, if needed, further advanced or positioned and appropriately secured in the trachea.

Alternatively, the steps can be further reduced to:
1. to 3. as above;
4. Upon locating the ETT into the trachea, the device 10 with bougie 18 connected thereto are removed from the ETT 12; and
5. The ETT 12 is, if needed, further advanced or positioned and appropriately secured in the trachea.

1. A connector device comprising:
a body portion having an opening for receiving a bougie or stylet; and
a tube engaging portion;
wherein a bougie or stylet locates into the opening of the body portion and the tube engaging portion engages with a tube or tube connector.

2-29. (canceled)

30. The connector device according to claim 1, further including a channel along and into a length of the connector device leading to a conduit or passage for receiving the bougie or stylet.

31. The connector device according to claim 1, further including a lever actuated mechanism for use in gripping and/or releasing the bougie or stylet.

32. The connector device according to claim 31, including a cam mechanism wherein a cam engages with the bougie or stylet to retain the bougie or stylet to the connector device.

33. The connector device according to claim 1, wherein the connector device grips the tube or tube connector by compression or interference fit.

34. The connector device according to claim 1, wherein the connector device is incorporated with the bougie or stylet by being moulded with or moulded to the bougie or stylet or adhered or bonded to the bougie or stylet.

35. The connector device according to claim 1, wherein the body portion is provided in two segments, a first segment having an opening to receive the bougie or stylet and a second segment being slidably moveable relative to first segment and tube engaging portion.

36. The connector device according to claim 35, wherein downward sliding of the second segment presses the second segment against the first segment, providing a compression fit to hold the bougie or stylet in place in the opening.

37. The connector device according to claim 35, wherein the second segment has a flange extending outwardly therefrom, whereby pushing or pulling of the flange causes sliding of the second segment relative to the first segment.

38. The connector device according to claim 1, further including one or more septations or divisions forming one or more respective passages through or one or more chambers within the connector device.

39. The connector device according to claim 1, further including at least one grip for assisting removal of the connector device from the tube or the tube connector.

40. The connector device according to claim 39, wherein the grip includes one or more of, or any two of, a loop, a handle, a projection, a textured surface or a flange extending outwardly from an end of the body portion distal to the tube engaging portion.

41. The connector device according to claim 1, further including securing means to limit movement of the bougie or stylet in longitudinal and/or rotational planes relative to the tube or the tube connector.

42. The connector device according to claim 41, wherein the securing means is operated or activated to hold the bougie or stylet in position relative to the connector device and the tube or the tube connector, limiting movement of the bougie or stylet in longitudinal and/or rotational planes relative to the respective tube or tube connector.

43. The connector device according to claim 1, wherein the tube is an endotracheal tube (ETT).

44. The connector device according to claim 1, wherein the opening of the body portion is shaped to receive a longitudinal mid-portion of the bougie or stylet.

45. The connector device according to claim 1, further including a port for connection of a tube to deliver gas into or through the connector device or to ventilate gas out of the connector device.

46. The connector device according to claim 1, wherein the connector includes an engaging portion to engage with the tube end or tube connector of an endotracheal tube (ETT), wherein the connector device engaging portion fits over or into, or both over and into, the tube end or the tube connector of the ETT, and the connector device provides a passage for the bougie or stylet to pass into the ETT.

47. The connector device according to claim 1, wherein the body portion is tapered or frusto-conical.

48. The connector device according to claim 1, wherein the connector device grips the bougie or stylet by compression or interference fit.

49. The connector device according to claim 1, further including securing means to limit movement of the bougie or stylet in longitudinal and/or rotational planes relative to the connector device.

50. The connector device according to claim 1, the connector device being in two halves that close together to grip the bougie or stylet.

51. The connector device according to claim 50, wherein the two halves are hinged at a join.
